(19) United States
(12) Patent Application Publication
(10) Pub. No.: US 2019/0307698 A1
(43) Pub. Date: Oct. 10, 2019

(54) DIRECTLY COMPRESSIBLE MATRIX FOR THE PRODUCTION OF TABLETS HAVING EXTENDED RELEASE OF ACTIVE PHARMACEUTICAL INGREDIENT

(71) Applicant: Merck Patent GmbH, Darmstadt (DE)

(72) Inventors: Gudrun BIRK, Darmstadt (DE); Guenter MODDELMOG, Reinheim (DE); Thorsten WEDEL, Stockstadt/Rhein (DE)

(73) Assignee: Merck Patent GmbH, Darmstadt (DE)

(21) Appl. No.: 16/469,332

(22) PCT Filed: Dec. 11, 2017
(86) PCT No.: PCT/EP2017/082122

§ 371 Date: Jun. 13, 2019

(30) Foreign Application Priority Data
Dec. 14, 2016 (EP) 16204112.3

(51) Int. Cl. A61K 9/20; A61K 9/14; A61K 31/138

(52) U.S. Cl. CPC A61K 9/2095; A61K 9/2054; A61K 31/138; A61K 9/14; A61K 9/2027

(57) ABSTRACT

The present invention relates to tablets having extremely long release of active pharmaceutical ingredient, to the particular composition thereof and to the production thereof.

The present invention relates to tablets having extremely long release of active pharmaceutical ingredient, to the composition thereof and to the production thereof.

### PRIOR ART

Polyvinyl alcohols (PVAs) are synthetic polymers which are available in various grades, in particular with respect to degree of polymerisation and viscosity. The use of relatively high-viscosity and also pharmacopoeia-compliant grades, such as PVA 26-88 and especially PVA 40-88, is particularly interesting for the formulation and production of so-called matrix retard tablets. The active pharmaceutical ingredient is released from these tablets in the gastrointestinal tract (GI tract) with a delay in a controlled manner over several hours with the aim of ensuring a very constant level of active pharmaceutical ingredient in the blood over a long period and thus improving the therapeutic effect and also patient compliance. This controlled release of active pharmaceutical ingredient is achieved by the swelling of the PVA after contact with aqueous media, such as, for example, the physiological fluids in the GI tract, with the active pharmaceutical ingredient being released into the medium in a delayed manner by diffusion from the gel layer which forms.

### OBJECT OF THE PRESENT INVENTION

Parteck® SRP 80, a commercially available polyvinyl alcohol grade which is a PVA 40-88 which has been optimised with respect to compressibility and release of active pharmaceutical ingredient, generally exhibits cumulative release of about 10 to 12 hours (90 to 100% final release of active pharmaceutical ingredient) in the various in vitro models and depending on the active pharmaceutical ingredient to be retarded. However, some users would like even more retarded in vitro release. It has to date usually not been possible to achieve such extremely long retardation of the release of active pharmaceutical ingredient with PVA 40-88 polyvinyl alcohol grades, even if the PVA content in the tablet recipes is increased. It is therefore an object of the present invention to increase the duration of release of active pharmaceutical ingredient from corresponding tablet formulations to more than 12 hours by suitable measures.

A further object consists in providing a pulverulent mixture containing active pharmaceutical ingredient with the above-mentioned, optimised PVA grades (PVA 40-88) as excipient material for the production of tablets containing active pharmaceutical ingredient which furthermore has a good flow and compression properties in order to be able to employ it also in direct-compression processes for the rapid and uncomplicated formulation of tablets having “extremely” retarded release of active pharmaceutical ingredient.

In patent applications WO 2016/015812 A1, WO 2016/015813 A1 and WO 2016/015814 A1, it was found that co-mixtures of ground polyvinyl alcohols (PVAs) of specific particle sizes with microcrystalline celluloses (MCCs) of specific particle sizes result in pulverulent pre-mixtures which have good compressibility.

In addition, the two patent applications with the application numbers PCT/EP2016/001430 and PCT/EP2016/001431 describe that matrix retard tablets containing active pharmaceutical ingredient which, besides the good tablet formulation properties, release the active ingredient over a period of 12 hours while exhibiting a cumulative in vitro release of active ingredient of 80 to 100% can be produced using these co-mixtures. In addition, the release of active ingredient from such formulations is, over broad ranges, virtually independent of the pressing forces used for the production of the tablets and the different tablet hardnesses resulting therefrom. Furthermore, it is shown in these applications that for these tablets the release of active ingredient is substantially independent of the pH in the range from pH 1 to 7 and the alcohol content (0 to 40% by vol.) of the release media. These are all factors which are prerequisites for the prevention of possible “dose-dumping” effects.

However, specific applications require even more pronounced retardation with even further delayed in vitro release of active pharmaceutical ingredient than has been found in the above-mentioned applications. In some recipes of matrix retard tablets, however, this aim cannot be achieved by a simple increase in the amounts of PVA present in the tablets. This is also dependent on further factors, such as, for example, the type and amount of the active pharmaceutical ingredient per tablet. It is therefore desirable also to be able to provide a suitable solution for such cases.

### BRIEF DESCRIPTION OF THE INVENTION

It has now been found that combinations of PVAs with microcrystalline cellulose (MCC) and hydroxypropylmethylcelluloses (HPMCs) of various viscosities exhibit good compression properties and greatly retarded in vitro release of active pharmaceutical ingredient.

Thus, the cumulative release of active pharmaceutical ingredient in a retarded 160 mg propranolol tablet recipe can be extended significantly beyond 12 hours. Surprisingly, this effect can be achieved with only small amounts of HPMC in the recipe. The experiments have shown that this effect is only dependent to a limited extent on the viscosity of the HPMC grades used. Synergistic interactions between the PVAs present and the HPMCs are evidently involved, since even the addition of small amounts of HPMC considerably retards the in vitro release.

If, for example, Parteck® SRP 80 (PVA 40-88 having a specific particle size distribution) as excipient material is combined with 5 to 10% of HPMC K4M (apparent viscosity according to the EP: 2663-4970 mPa·s) or K100M (apparent viscosity according to the EP: 75000-140000 mPa·s), the cumulative (90 to 100%) in vitro release of active pharmaceutical ingredient in a retarded 160 mg propranolol retard tablet can be extended into a range from about 17 to 32 hours. With a further increase in the amounts of HPMC, it is even possible to achieve cumulative API release times of more than 32 hours. This is comparable with the release of propranolol from the two “pure” (but) 32% HPMC recipes (without PVA) at the same point in time, although it should be taken into account that, owing to the very low bulk/tapped weight of the “pure” HPMC recipes, the target weight of the propranolol tablet of 500 mg (for the same dimensions) cannot be achieved, i.e. a lower active pharmaceutical ingredient content was obtained in the “same” recipe.

### DETAILED DESCRIPTION OF THE INVENTION

Attempts to extend the duration of the release of active pharmaceutical ingredient from tablets in which PVA serves as excipient by increasing the content of PVA in the formulation have not led to a positive result. Attempts have therefore been made to modify the properties of the tablet matrix in such a way that, in the presence of aqueous media, as is the case in the GI tract, both the dissolution rate of the matrix itself is retarded further, but at the same time diffusion of the active pharmaceutical ingredient out of the tablet is also considerably slowed.

Experiments have been carried out to investigate the influence on the release duration if the percentage amounts of the excipient materials PVA and MCC, which have previously been found to be effective, to one another are varied. Since these did not show adequate extension of the release, it was attempted to extend the duration of the release of active pharmaceutical ingredient by addition of a further suitable retard component in the tableting matrix.

These experiments have shown that the use of co-mixtures of polyvinyl alcohols (PVAs) and specific microcrystalline celluloses (MCCs) with addition of further hydrophilic polymers, in particular hydroxypropylmethyl-celluloses of various viscosities, enables the in vitro release of active pharmaceutical ingredient to be retarded even further.

In addition, the experimental data also enable it to be shown that the compressibility of such mixtures, consisting of the three components PVA, MCC and HPMC, and the formulation properties of the tablets resulting from them are not impaired. In particular, it has been found that matrix tablets of this type obtained by a simple direct compression process have even greater retardation of the release of active pharmaceutical ingredient.

In this way, the formulation chemist is able to influence the in vitro release profiles of retard tablets and considerably to extend the release of the active pharmaceutical ingredient in a simple process (direct compression) by simple mixing of an active pharmaceutical ingredient (API) with a PVA/HPMC/MCC pre-mixture. Given suitable mixing ratios of three components, the term “extreme” extension of the release of active pharmaceutical ingredient can be used. The considerably higher bulk and tapped densities of the PVA/HPMC/MCC combinations, which enable tablets having smaller dimensions with the same weight to be obtained, are particularly advantageous compared with the recipes based solely on HPMC.

The experiments have shown that, using the co-mixtures according to the invention, consisting of the three components PVA, MCC and HPMC in various mixing ratios, enables the production of retard tablets which
1. can be obtained particularly quickly by a simple direct compression process without complex granulation processes,
2. can be compressed even at low pressing forces to give tablets of high hardnesses and low friability, and which
3. can be produced in a simple process and (here: propranolol tablets) exhibit particularly extended or particularly strongly retarded in vitro release of active pharmaceutical ingredient.

This co-combination of PVA, MCC and HPMC thus provides the drug developer with a fast way of producing active pharmaceutical ingredient tablets having an extremely retarded in vitro release profile, and formulating an active pharmaceutical ingredient in an uncomplicated mixing process with the pre-mixture consisting of the above three components, and the desired tablets by subsequent direct compression.

In order to carry out the invention described here, the following steps are necessary:
1. Preparation of the co-mixtures analogously to examples A2J given below, comprising M CC with various amounts and grades of ground PVA and HPMC, as indicated in the tables under “Characterisation of the raw materials used”, and determination of the powder characteristics.
The preparation of the comparative mixtures without PVA or without HPMC (Comparisons 1 to 4) and the determination of the powder characteristics are carried out with the compositions as indicated in the tables under “Characterisation of the raw materials used”.
2. Mixing of these co-mixtures with the active pharmaceutical ingredient, here by way of example with propranolol HCl, and further additives and compression at pressing forces of 5, 10, 20 and 30 kN with subsequent pharmaceutical characterisation of the tablets obtained.
3. Measurement of the in vitro release of propranolol HCl in phosphate buffer pH 6.8 over 12 or 42 hours—testing of the tablets obtained at a pressing force of 20 kN.

The results of the experiments carried out, as described in the examples given, show that tablets having considerably extended release of active pharmaceutical ingredient are obtained.

The examples given below disclose methods and conditions for the preparation of the retard formulations according to the invention containing active pharmaceutical ingredient having extremely extended release of active ingredient. It is self-evident to the person skilled in the art that methods for the preparation of the pre-mixtures and the tablet matrices other than those described here are also available.

The examples show the particular advantages of these PVA/MCC/HPMC combinations.

The present description enables the person skilled in the art to apply the invention comprehensively. Even without further comments, it is therefore assumed that a person skilled in the art will be able to utilise the above description in the broadest scope.

If anything is unclear, it goes without saying that the publications and patent literature cited should be consulted. Accordingly, these documents are regarded as part of the disclosure of the present description.

For better understanding and illustration of the invention, examples are given below which are within the scope of protection of the present invention. These examples also serve to illustrate possible variants. Owing to the general validity of the inventive principle described, however, the examples are not suitable for reducing the scope of protection of the present application to these alone.

Furthermore, it goes without saying for the person skilled in the art that, both in the examples given and also in the remainder of the description, the component amounts present in the compositions always only add up to 100% by weight or mol-%, based on the composition as a whole, and cannot exceed this, even if higher values could arise from the per cent ranges indicated. Unless indicated otherwise, % data are thus regarded as % by weight or mol-%, with the exception of ratios, which are reproduced in volume figures.

The temperatures given in the examples and the description as well as in the claims are in ° C.

### EXAMPLES

The conditions for the production and for analytical and pharmaceutical testing are given in the examples. The retard tablets are produced by direct compression. In this connection, very particular preference is given to co-mixtures consisting of the pulverulent PVAs 40-88 (Parteck® SRP 80, Merck KGaA, Germany) or 26-88 with the HPMCs Methocel® K4M and K100M (both DOW) in combination with MCC Vivapur® 102 (JRS), where the components PVA, MCC and HPMC are preferably used in the weight ratios from 50:45.5:4.5 to 50:15:35 and are employed as preferred retardation matrices.

### Instruments and Methods for the Characterisation of the Material Properties

1. Bulk density: in accordance with DIN EN ISO 60:1999 (German version)
quoted in “g/ml”

2. Tapped density: in accordance with DIN EN ISO 787-11:1995 (German version)
quoted in “g/ml”

3. Angle of repose (of the raw materials employed): in accordance with DIN ISO 4324:1983 (German version)
quoted in “degrees”

4. Surface area determined by the BET method: evaluation and procedure in accordance with the literature “Adsorption of Gases in Multimolecular Layers” by S. Brunauer et al. (Journal of American Chemical Society, 60, 1938)
Instrument: ASAP 2420 Micromeritics Instrument Corporation (USA); nitrogen; sample weight: about 3.0000 g; heating: 50° C. (5 h); heating rate 3 K/min; arithmetic mean from three determinations quoted

5. Particle size determination by laser diffraction with dry dispersal: Master-sizer 2000 with Scirocco 2000 dispersion unit (Malvern Instruments Ltd., UK), determinations at a counterpressure of 1, 2 and 3 bar; Fraunhofer evaluation; dispersant RI: 1.000, obscuration limits: 0.1-10.0%, tray type: general purpose, background time: 7500 msec, measurement time: 7500 msec, procedure in accordance with ISO 13320-1 and the information in the technical manual and specifications from the instrument manufacturer; quoted in % by vol.

6. Angle of repose, angle of fall, angle of difference and angle of spatula (of the pre-mixtures of PVA, HPMC and MCC; Examples A to J or Comparisons 1 to 4):
Measurement in a Hosokawa powder tester model PT-X (HOSOKAWA Alpine, Augsburg, Germany) in accordance with the user manual or menu guide on the instrument during the measurement operation
all figures quoted in “degrees”
Sieve for PT-X: aperture: 1.7 mm, wire dia: 0.8 mm, S/N: XS1700-205 PT-X attachment for determination of angle of repose and angle of spatula

7. Tableting tests:
The mixtures in accordance with the compositions indicated in the experimental part are mixed for 5 minutes in a sealed stainless-steel container (capacity: about 2 I, height: about 19.5 cm, diameter: about 12 cm outside dimension) in a laboratory tumble mixer (Turbula T2A, Willy A. Bachofen, Switzerland).
The magnesium stearate employed is Parteck® LUB MST (vegetable magnesium stearate) EMPROVE® exp Ph. Eur., BP, JP, NF, FCC Article No. 1.00663 (Merck KGaA, Germany) which has been passed through a 250 μm sieve.
The compression to give 500 mg or 450 mg tablets (11 mm punch, round, flat, with bevel edge) is carried out in a Korsch EK 0-DMS instrumented eccentric tableting machine (Korsch, Germany) with the Catman 5.0 evaluation system (Hottinger Baldwin Messtechnik—HBM, Germany).
Depending on the pressing force tested (nominal settings: ˜5, ˜10, ˜20 and ˜30 kN; the effectively measured actual values are indicated in the examples), at least 100 tablets are produced for evaluation of the pressing data and determination of the pharmaceutical characteristics.

Tablet hardnesses, diameters and heights: Erweka Multicheck® 5.1 (Erweka, Germany); average data (arithmetic means) from in each case 20 tablet measurements per pressing force. The measurements are carried out one day after tablet production.

Tablet abrasion: TA420 friability tester (Erweka, Germany); instrument parameters and performance of the measurements in accordance with Ph. Eur. 7th Edition “Friability of Uncoated Tablets”. The measurements are carried out one day after tablet production.

Tablet weight: Average (arithmetic mean) from the weighing of 20 tablets per pressing force: Multicheck® 5.1 (Erweka, Germany) with Sartorius CPA 64 balance (Sartorius, Germany). The measurements are carried out one day after tablet production.

8. Propranolol release test: The tablets containing propranolol HCl (pressed with a pressing force of 20 kN) are measured in an in vitro release apparatus from ERWEKA (Heusenstamm, Germany) using the “Apparatus 2 (Paddle Apparatus)” described in Ph. Eur. 8.4 under 2.9.3. “Dissolution test for solid dosage forms” and under the conditions described therein (Ph. Eur.=European Pharmacopoeia). The sampling is carried out automatically via a hose pump system with subsequent measurement in a Lambda® 35 photometer (Perkin Elmer, USA) and a flow cell.

### Measurement Apparatuses and Measurement Parameters

ERWEKA DT70 release apparatus fitted with Apparatus 2 (Paddle Apparatus in accordance with Ph. Eur.), ERWEKA, Germany

Temperature: 37° C.+/−0.5° C.

Speed of rotation of the paddle: 50 rpm

Release medium: 900 ml of phosphate buffer pH 6.8 in accordance with Ph. Eur.

Total running time of the measurements: 12 or 42 hours (with sampling after 15, 30, 45, 60 minutes or hourly thereafter up to 12 hours or additionally after a total running time of 17, 22, 27, 32, 37 and 42 hours (in the tables and graphs, the data for the 15, 30 and 45 minute samples are not shown)—Exception: in the case of the 42 hour measurements, no samples are taken after a release time of 7 or 9 hours

Hose pump with sampling: Ismatec IPC, model ISM 931; App. No. 12369-00031

Lambda® 35 photometer, Perkin Elmer, Germany

Measurement at 214 nm in a 0.5 mm flow cell

Evaluation via Dissolution Lab Software Version 1.1, ERWEKA, Germany

### Characterisation of the Raw Materials Used

1. PVA 40-88 and PVA 26-88:
1.1 Raw materials for grinding
1.1.1. PVA 26-88: polyvinyl alcohol 26-88, suitable for use as excipient EMPROVE® exp Ph. Eur., USP, JPE, Article No. 1.41352, Merck KGaA, Darmstadt, Germany
1.1.2. PVA 40-88: polyvinyl alcohol 40-88, suitable for use as excipient EMPROVE® exp Ph. Eur., USP, JPE, Article No. 1.41353, Merck KGaA, Darmstadt, Germany

These PVA grades are in the form of coarse particles with a size of several millimetres which cannot be employed in this form as a directly compressible tableting matrix.

The coarse particles do not allow reproducible filling of the dies and thus also do not allow a constant tablet weight at the high rotational speeds of the (rotary) tableting machines. In addition, only fine-grained PVAs are able to ensure homogeneous distribution of the active pharmaceutical ingredient in the tablet—without the occurrence of separation effects. This is absolutely necessary for ensuring individual dosage accuracy of the active pharmaceutical ingredient (content uniformity) in each tablet produced. In addition, only a fine-grained PVA can also ensure the homogeneous gel formation throughout the tablet body that is necessary for reproducible retardation.

For these reasons, the above-mentioned coarse-grained PVA grades must be comminuted, i.e. ground, before use as directly compressible retardation matrices.
1.2 Ground PVA grades
1.2.1 Ground PVA 26-88, from polyvinyl alcohol 26-88, Article No. 1.41352, batch F1842262 having the average particle-size fractions Dv50 (laser diffraction; dry dispersal): Dv50 80-90 μm
1.2.2 Ground PVA 40-88, from polyvinyl alcohol 40-88 Article No. 1.41353, batch F1885763 having the average particle-size fractions Dv50 (laser diffraction; dry dispersal): Dv50 70-80 μm

### Grinding

The grinding of the PVA grades is carried out in an Aeroplex® 200 AS spiral jet mill from Hosokawa Alpine, Augsburg, Germany, under liquid nitrogen as cold grinding at 0° C. to minus 30° C. The desired particle size is produced empirically, in particular by variation of the grinding temperature, i.e. the grinding conditions are varied by ongoing in-process controls of the particle size until the desired particle size fraction is obtained.

The resultant product properties of the ground PVA grades, in particular the powder characteristics, such as bulk density, tapped density, angle of repose, BET surface area, BET pore volume as well as the particle size distributions, are evident from the following tables:

Bulk Density, Tapped Density, Angle of Repose, BET Surface Area, BET Pore Volume:

(details on the measurement methods, see under Methods)

| | Bulk | Tapped | Angle of | BET | BET |
|---|---|---|---|---|---|
| | density | density | repose | surface area | pore volume |
| Sample | (g/ml) | (g/ml) | (°) | (m²/g) | (cm³/g) |
| PVA 26-88 | 0.51 | 0.70 | 36.7 | 0.35 | 0.0019 |
| PVA 40-88 | 0.54 | 0.75 | 33.9 | 0.33 | 0.0020 |

Particle Distribution Determined by Laser Diffraction with Dry Dispersal (1 Bar Counterpressure):

Figures in μm (details on the measurement method, see under Methods)

| Sample | Dv5 | Dv10 | Dv20 | Dv25 | Dv30 | Dv50 | Dv75 | Dv90 | Dv95 |
|---|---|---|---|---|---|---|---|---|---|
| PVA 26-88 | 17.39 | 24.78 | 38.52 | 45.59 | 52.97 | 87.60 | 161.70 | 285.80 | 526.73 |
| PVA 40-88 | 16.07 | 22.39 | 35.62 | 42.01 | 48.44 | 76.82 | 129.95 | 203.89 | 324.47 |

Particle Distribution Determined by Laser Diffraction with Dry Dispersal (2 Bar Counterpressure):

Figures in μm (details on the measurement method, see under Methods)

| Sample | Dv5 | Dv10 | Dv20 | Dv25 | Dv30 | Dv50 | Dv75 | Dv90 | Dv95 |
|---|---|---|---|---|---|---|---|---|---|
| PVA 26-88 | 16.15 | 23.53 | 37.22 | 44.26 | 51.56 | 85.05 | 151.3 | 240.02 | 305.79 |
| PVA 40-88 | 15.35 | 22.91 | 36.08 | 42.38 | 48.71 | 76.62 | 129.10 | 197.91 | 253.89 |

Particle Distribution Determined by Laser Diffraction with Dry Dispersal (3 Bar Counterpressure):

Figures in μm (details on the measurement method, see under Methods)

| Sample | Dv5 | Dv10 | Dv20 | Dv25 | Dv30 | Dv50 | Dv75 | Dv90 | Dv95 |
|---|---|---|---|---|---|---|---|---|---|
| PVA 26-88 | 15.99 | 23.44 | 37.29 | 44.35 | 51.65 | 84.88 | 150.53 | 237.38 | 299.34 |
| PVA 40-88 | 15.12 | 22.65 | 35.82 | 42.11 | 48.42 | 76.09 | 127.20 | 192.84 | 240.56 |

2. Microcrystalline Celluloses (MCCs)

Vivapur® Type 102 Premium, microcrystalline cellulose, Ph. Eur., NF, JP, JRS Pharma, Rosenberg, Germany

Particle distribution determined by laser diffraction with dry dispersal (1 bar counterpressure):

Figures in μm (details on the measurement method, see under Methods)

| Sample | Dv10 | Dv20 | Dv25 | Dv30 | Dv50 | Dv75 | Dv90 |
|---|---|---|---|---|---|---|---|
| Vivapur ® 102 | 31.56 | 53.04 | 66.00 | 79.89 | 135.87 | 215.53 | 293.94 |

Particle distribution determined by laser diffraction with dry dispersal (2 bar counterpressure):

Figures in μm (details on the measurement method, see under Methods)

| Sample | Dv10 | Dv20 | Dv25 | Dv30 | Dv50 | Dv75 | Dv90 |
|---|---|---|---|---|---|---|---|
| Vivapur ® 102 | 27.55 | 45.97 | 57.41 | 70.40 | 127.29 | 208.92 | 288.93 |

Particle distribution determined by laser diffraction with dry dispersal (3 bar counterpressure):

Figures in μm (details on the measurement method, see under Methods)

| Sample | Dv10 | Dv20 | Dv25 | Dv30 | Dv50 | Dv75 | Dv90 |
|---|---|---|---|---|---|---|---|
| Vivapur ® 102 | 23.61 | 38.84 | 48.19 | 59.22 | 114.76 | 198.37 | 278.99 |

3. Hydroxypropylmethylcelluloses (HPMCs)
HPMC K100M: Methocel® K100M Premium CR Hydroxypropyl methylcellulose USP, EP, JP; 75000-140000 mPa·s (apparent viscosity: Brookfield, 2% in water, 20° C.); DOW CHEMICAL COMPANY, U.S.A.
HPMC K4M: Methocel® K4M Premium CR Hydroxypropyl methylcellulose USP, EP, JP; 2663-4970 mPa·s (apparent viscosity: Brookfield, 2% in water, 20° C.); DOW CHEMICAL COMPANY, U.S.A.

Particle distribution determined by laser diffraction with dry dispersal (1 bar counterpressure):

Figures in μm (details on the measurement method, see under Methods)

| Sample | Dv5 | Dv10 | Dv20 | Dv25 | Dv30 | Dv50 | Dv75 | Dv90 | Dv95 |
|---|---|---|---|---|---|---|---|---|---|
| HPMC K100M | 17.32 | 25.51 | 37.76 | 43.46 | 49.21 | 75.43 | 128.50 | 197.53 | 244.88 |
| HPMC K4M | 15.94 | 24.88 | 40.15 | 47.71 | 55.54 | 92.57 | 166.70 | 257.99 | 319.34 |

Particle distribution determined by laser diffraction with dry dispersal (2 bar counterpressure):

Figures in μm (details on the measurement method, see under Methods)

| Sample | Dv5 | Dv10 | Dv20 | Dv25 | Dv30 | Dv50 | Dv75 | Dv90 | Dv95 |
|---|---|---|---|---|---|---|---|---|---|
| HPMC K100M | 16.00 | 24.32 | 36.49 | 42.07 | 47.67 | 73.05 | 124.57 | 192.55 | 239.26 |
| HPMC K4M | 14.57 | 23.35 | 38.09 | 45.26 | 52.63 | 87.32 | 157.32 | 243.26 | 299.93 |

Particle distribution determined by laser diffraction with dry dispersal (3 bar counterpressure):

Figures in μm (details on the measurement method, see under Methods)

| Sample | Dv5 | Dv10 | Dv20 | Dv25 | Dv30 | Dv50 | Dv75 | Dv90 | Dv95 |
|---|---|---|---|---|---|---|---|---|---|
| HPMC K100M | 14.72 | 23.01 | 35.03 | 40.47 | 45.91 | 70.32 | 119.45 | 184.52 | 229.51 |
| HPMC K4M | 13.66 | 22.26 | 36.73 | 43.74 | 50.93 | 84.61 | 152.98 | 238.71 | 296.37 |

4. Other Materials
4.1 Propranolol HCl BP, EP, USP Batch No. M150101 (Changzhou Yabang Pharmaceutical Co., LTD., China)
4.2 Parteck® LUB MST (vegetable magnesium stearate) EMPROVE® exp Ph. Eur., BP, JP, NF, FCC
   Article No. 1.00663 (Merck KGaA, Germany)
4.3 Colloidal silicon dioxide, highly disperse, suitable for use as excipient EMPROVE® exp Ph. Eur., NF, JP, E 551
   Article No. 1.13126 (Merck KGaA, Germany)

5. Compositions and preparations of Examples A to J or Comparisons 1 to 4
(figures in “% by weight”)

a) PVA 40-88, MCC and HPMC K100M mixtures:
Examples A to D and Comparisons 1 and 2

| | Exam- | Exam- | Exam- | Exam- | Compar- | Compar- |
|---|---|---|---|---|---|---|
| | ple A | ple B | ple C | ple D | ison 1 | ison 2 |
| PVA | 50 | 50 | 50 | 50 | 50 | — |
| 40-88 | | | | | | |
| MCC | 45.5 | 42.5 | 35 | 15 | 50 | 50 |
| HPMC | 4.5 | 7.5 | 15 | 35 | — | 50 |
| K100M | | | | | | |

b) PVA 40-88, MCC and HPMC K4M mixtures:
Examples E to H and Comparison 3

| | Exam- | Exam- | Exam- | Exam- | Compar- | Compar- |
|---|---|---|---|---|---|---|
| | ple E | ple F | ple G | ple H | ison 1 | ison 3 |
| PVA | 50 | 50 | 50 | 50 | 50 | — |
| 40-88 | | | | | | |
| MCC | 45.5 | 42.5 | 35 | 15 | 50 | 50 |
| HPMC | 4.5 | 7.5 | 15 | 35 | — | 50 |
| K4M | | | | | | |

c) PVA 26-88, MCC and HPMC K100M mixtures:
Examples I and J and Comparison 4

| | Example I | Example J | Comparison 2 | Comparison 4 |
|---|---|---|---|---|
| PVA 26-88 | 50 | 50 | — | 50 |
| MCC | 35 | 15 | 50 | 50 |
| HPMC K100M | 15 | 35 | 50 | — |

### Experimental Results

### For Step 1: Preparation and Pharmaceutical Characterisation of the Co-Mixtures Examples A to J and Comparisons 1 to 4

### Preparation of 1 kg of the Co-Mixtures of Examples A to J and Comparisons 1 to 4

### Compositions of the Mixtures in the Tables in “g”

TABLE 1a
PVA 40-88, MCC and HPMC K100M mixtures:
Examples A to D and Comparisons 1 and 2

| | Exam- | Exam- | Exam- | Exam- | Compar- | Compar- |
|---|---|---|---|---|---|---|
| | ple A | ple B | ple C | ple D | ison 1 | ison 2 |
| PVA | 500 | 500 | 500 | 500 | 500 | — |
| 40-88 | | | | | | |
| MCC | 455 | 425 | 350 | 150 | 500 | 500 |
| HPMC | 45 | 75 | 150 | 350 | — | 500 |
| K100M | | | | | | |

TABLE 1b
PVA 40-88, MCC and HPMC K4M mixtures:
Examples E to H and Comparisons 1 and 3

| | Exam- | Exam- | Exam- | Exam- | Compar- | Compar- |
|---|---|---|---|---|---|---|
| | ple E | ple F | ple G | ple H | ison 1 | ison 3 |
| PVA | 500 | 500 | 500 | 500 | 500 | — |
| 40-88 | | | | | | |
| MCC | 455 | 425 | 350 | 150 | 500 | 500 |
| HPMC | 45 | 75 | 150 | 350 | — | 500 |
| K4M | | | | | | |

TABLE 1c
PVA 26-88, MCC and HPMC K100M mixtures: Examples I and
J and Comparisons 2 and 4

| | Example I | Example J | Comparison 2 | Comparison 4 |
|---|---|---|---|---|
| PVA 26-88 | 500 | 500 | — | 500 |
| MCC | 350 | 150 | 500 | 500 |
| HPMC K100M | 150 | 350 | 500 | — |

Preparation of the mixtures: The components mentioned in Examples A to J and Comparisons 1 to 4 are weighed out directly, without pre-treatment, into a drum hoop mixer (stainless-steel drum having a diameter of about 25 cm, a height of about 13 cm and a volume of about 6 I) and mixed for 5 min. in a drum hoop mixer (Elte 650, Engelsmann AG, Ludwigshafen, Germany) at setting 6 with a speed of about 28 revolutions/minute. In each case 1 kg of said mixtures A to J and 1 to 4 are prepared.

### Powder Characteristics of Examples A to J and Comparisons 1 to 4

TABLE 2a
PVA 40-88, MCC and HPMC K100M mixtures:
Examples A to D and Comparisons 1 and 2

| | Exam- | Exam- | Exam- | Exam- | Comp. | Comp. |
|---|---|---|---|---|---|---|
| | ple A | ple B | ple C | ple D | 1 | 2 |
| Bulk density | 0.41 | 0.41 | 0.41 | 0.41 | 0.41 | 0.33 |
| (g/ml) | | | | | | |
| (DIN ISO 60) | | | | | | |
| Tapped density | 0.59 | 0.59 | 0.60 | 0.60 | 0.59 | 0.49 |
| (g/ml) | | | | | | |
| (DIN EN ISO | | | | | | |
| 787-11) | | | | | | |
| Angle of | 38.2 | 40.2 | 38.3 | 38.2 | 38.9 | 40.6 |
| repose (°) | | | | | | |
| (HOSOKAWA | | | | | | |
| PT-X) | | | | | | |
| Angle of | 20.8 | 21.9 | 21.5 | 21.3 | 24.1 | 23.3 |
| fall (°) | | | | | | |
| (HOSOKAWA | | | | | | |
| PT-X) | | | | | | |
| Angle of | 17.4 | 18.3 | 16.8 | 16.9 | 14.8 | 17.3 |
| difference (°) | | | | | | |
| (HOSOKAWA | | | | | | |
| PT-X) | | | | | | |
| Angle of | 52.2 | 50.0 | 53.2 | 51.6 | 50.8 | 52.1 |
| spatula (°) | | | | | | |
| (HOSOKAWA | | | | | | |
| PT-X) | | | | | | |

TABLE 2b
PVA 40-88, MCC and HPMC K4M mixtures:
Examples E to H and Comparisons 1 and 3

| | Exam- | Exam- | Exam- | Exam- | Comp. | Comp. |
|---|---|---|---|---|---|---|
| | ple E | ple F | ple G | ple H | 1 | 3 |
| Bulk density | 0.40 | 0.42 | 0.41 | 0.40 | 0.41 | 0.33 |
| (g/ml) | | | | | | |
| (DIN ISO 60) | | | | | | |
| Tapped density | 0.59 | 0.59 | 0.60 | 0.60 | 0.59 | 0.50 |
| (g/ml) | | | | | | |
| (DIN EN ISO | | | | | | |
| 787-11) | | | | | | |
| Angle of | 39.3 | 37.1 | 38.3 | 38.8 | 38.9 | 40.4 |
| repose (°) | | | | | | |
| (HOSOKAWA | | | | | | |
| PT-X) | | | | | | |
| Angle of | 20.4 | 18.8 | 19.7 | 21.2 | 24.1 | 22.3 |
| fall (°) | | | | | | |
| (HOSOKAWA | | | | | | |
| PT-X) | | | | | | |
| Angle of | 18.9 | 18.3 | 18.5 | 17.6 | 14.8 | 18.1 |
| difference (°) | | | | | | |
| (HOSOKAWA | | | | | | |
| PT-X) | | | | | | |
| Angle of | 50.9 | 49.5 | 50.6 | 53.2 | 50.8 | 52.1 |
| spatula (°) | | | | | | |
| (HOSOKAWA | | | | | | |
| PT-X) | | | | | | |

TABLE 2c
PVA 26-88, MCC und HPMC K100M mixtures:
Examples I and J and Comparisons 2 and 4

| | | | Com- | Com- |
|---|---|---|---|---|
| | Example I | Example J | parison 2 | parison 4 |
| Bulk density (g/ml) | 0.40 | 0.40 | 0.33 | 0.40 |
| (DIN ISO 60) | | | | |
| Tapped density (g/ml) | 0.58 | 0.58 | 0.49 | 0.58 |
| (DIN EN ISO 787-11) | | | | |
| Angle of repose (°) | 38.7 | 40.1 | 40.6 | 38.2 |
| (HOSOKAWA PT-X) | | | | |
| Angle of fall (°) | 20.4 | 22.6 | 23.3 | 21.9 |
| (HOSOKAWA PT-X) | | | | |
| Angle of difference (°) | 18.3 | 17.6 | 17.3 | 16.3 |
| (HOSOKAWA PT-X) | | | | |
| Angle of spatula (°) | 49.5 | 52.7 | 52.1 | 52.9 |
| (HOSOKAWA PT-X) | | | | |

All mixtures exhibit adequate powder characteristics and make them suitable for further processing in tablet recipes for direct compression.

The exceptions are the mixtures of MCC and HPMC (without PVA) in Comparisons 2 and 3, whose bulk and tapped densities are significantly lower than the PVA-containing co-mixtures. This property can result in metering problems (excessively low weight for the same tablet dimensions) or excessively large tablet dimensions (for the same weight).

### For Step 2: Composition, Preparation and Pharmaceutical Characterisation of the Propranolol Retard Tablets

### Preparation of 500 q of Ready-to-Press Mixture Using Examples A to J and Comparisons 1 to 4

TABLE 3a
Composition (in % by weight) of propranolol HCl retard tablets
using the pre-mixtures of Examples A to D (gives tablets
A to D) and Comparisons 1 and 2 (gives tablets 1 and 2)

| | Tab- | Tab- | Tab- | Tab- | Tab- | Tab- |
|---|---|---|---|---|---|---|
| | let A | let B | let C | let D | let 1 | let 2 |
| Propranolol HCl | 32.0 | 32.0 | 32.0 | 32.0 | 32.0 | 35.56 |
| Example A | 67.0 | — | — | — | — | — |
| Example B | — | 67.0 | — | — | — | — |
| Example C | — | — | 67.0 | — | — | — |
| Example D | — | — | — | 67.0 | — | — |
| Comparison 1 | — | — | — | — | 67.0 | — |
| Comparison 2 | — | — | — | — | — | 63.44 |
| Silicon dioxide | 0.5 | 0.5 | 0.5 | 0.5 | 0.5 | 0.5 |
| Magnesium stearate | 0.5 | 0.5 | 0.5 | 0.5 | 0.5 | 0.5 |

TABLE 3b
Composition (in % by weight) of propranolol HCl retard tablets
using the pre-mixtures of Examples E to H (gives tablets
E to H) and Comparisons 1 and 3 (gives tablets 1 and 3)

| | Tab- | Tab- | Tab- | Tab- | Tab- | Tab- |
|---|---|---|---|---|---|---|
| | let E | let F | let G | let H | let 1 | let 3 |
| Propranolol HCl | 32.0 | 32.0 | 32.0 | 32.0 | 32.0 | 35.56 |
| Example E | 67.0 | — | — | — | — | — |
| Example F | — | 67.0 | — | — | — | — |
| Example G | — | — | 67.0 | — | — | — |
| Example H | — | — | — | 67.0 | — | — |
| Comparison 1 | — | — | — | — | 67.0 | — |
| Comparison 3 | — | — | — | — | — | 63.44 |
| Silicon dioxide | 0.5 | 0.5 | 0.5 | 0.5 | 0.5 | 0.5 |
| Magnesium stearate | 0.5 | 0.5 | 0.5 | 0.5 | 0.5 | 0.5 |

TABLE 3c
Composition (in % by weight) of propranolol HCl retard tablets
using the pre-mixtures of Examples I and J (gives tablets I and
mparisons 2 and 4 (gives tablets 2 and 4)

| | Tablet I | Tablet J | Tablet 2 | Tablet 4 |
|---|---|---|---|---|
| Propranolol HCl | 32.0 | 32.0 | 35.56 | 32.0 |
| Example I | 67.0 | — | — | — |
| Example J | — | 67.0 | — | — |
| Comparison 1 | — | — | 63.44 | — |
| Comparison 3 | — | — | — | 67.0 |
| Silicon dioxide | 0.5 | 0.5 | 0.5 | 0.5 |
| Magnesium stearate | 0.5 | 0.5 | 0.5 | 0.5 |

Preparation of the mixtures: in each case 335 g of co-mixtures A to J and comparative mixtures 1 and 4 are mixed with 160 g of propranolol HCl and 2.5 g of highly disperse silicon dioxide in a Turbula® mixer for 5 minutes. The mixture is then passed through a 560 μm hand sieve.

After addition of 2.5 g of Parteck® LUB MST in each case, mixing is continued for a further 5 minutes, and the mixture is subsequently compressed in a Korsch EK 0-DMS eccentric press (Korsch AG, Berlin, Germany) to give tablets weighing 500 mg; this corresponds to 160 mg of propranolol HCl per tablet.

Exception: since Comparisons 2 and 3 have a bulk density which is too low for tableting to give a tablet weight of 500 mg in the tablet machine used, only 285.5 g of comparative mixtures 2 and 3 and 2.25 g of highly disperse silicon dioxide and 2.25 g of Parteck® LUB MST are weighed out for tablets 2 and 3. The tableting is carried out to a tablet weight of 450 mg; this corresponds to 160 mg of propranolol HCl per tablet.

The tablet characterisation is carried out with respect to the parameters tablet hardness, tablet weight, tablet height, tablet abrasion and ejection force required.

### Tablet Characterisation

TABLE 4a
Tableting data of propranolol HCl retard tablets using the
pre-mixtures of Examples A to D and Comparisons 1 and 2

| | A | | | | | | |
|---|---|---|---|---|---|---|---|
| Parameter | Nominal | Actual | B | C | D | E | F |
| Tablet A | 5 | 5.0 | 45 | 498.3 | 5.5 | 2.08 | 324 |
| | 10 | 10.3 | 116 | 502.8 | 5.0 | 0.08 | 547 |
| | 20 | 19.1 | 222 | 506.9 | 4.6 | 0.02 | 467 |
| | 30 | 30.9 | 288 | 504.0 | 4.5 | 0.00 | 425 |
| Tablet B | 5 | 4.5 | 42 | 491.8 | 5.4 | 7.55 | 230 |
| | 10 | 9.4 | 106 | 497.8 | 4.9 | 0.17 | 389 |
| | 20 | 19.6 | 206 | 489.6 | 4.5 | 0.22 | 403 |
| | 30 | 29.1 | 257 | 489.7 | 4.4 | 0.05 | 395 |
| Tablet C | 5 | 5.3 | 57 | 505.5 | 5.4 | 1.08 | 166 |
| | 10 | 9.4 | 115 | 496.5 | 4.9 | 0.19 | 247 |
| | 20 | 20.5 | 217 | 492.6 | 4.5 | 0.06 | 282 |
| | 30 | 29.2 | 255 | 492.9 | 4.4 | 0.06 | 281 |
| Tablet D | 5 | 5.4 | 57 | 496.6 | 5.4 | 1.89 | 174 |
| | 10 | 10.5 | 138 | 500.1 | 4.9 | 0.13 | 247 |
| | 20 | 21.2 | 225 | 488.1 | 4.5 | 0.07 | 252 |
| | 30 | 32.3 | 258 | 488.2 | 4.4 | 0.06 | 254 |
| Tablet 1 | 5 | 4.7 | 48 | 499.7 | 5.3 | 1.09 | 160 |
| | 10 | 10.1 | 125 | 504.3 | 4.8 | 0.01 | 267 |
| | 20 | 19.8 | 220 | 501.5 | 4.4 | 0.00 | 311 |
| | 30 | 28.8 | 270 | 506.2 | 4.4 | 0.01 | 322 |
| Tablet 2 | 5 | 5.1 | 71 | 449.2 | 4.8 | 0.28 | 298 |
| | 10 | 9.9 | 164 | 451.1 | 4.3 | 0.03 | 404 |
| | 20 | 18.6 | 265 | 450.0 | 4.0 | 0.04 | 370 |
| | 30 | 32.2 | 332 | 453.3 | 4.0 | 0.04 | 376 |

Parameters:
A: Pressing force [kN]
B: Tablet hardness after 1 day [N]
C: Tablet weight [mg]
D: Tablet height [mm]
E: Abrasion [%]
F: Ejection force (N)

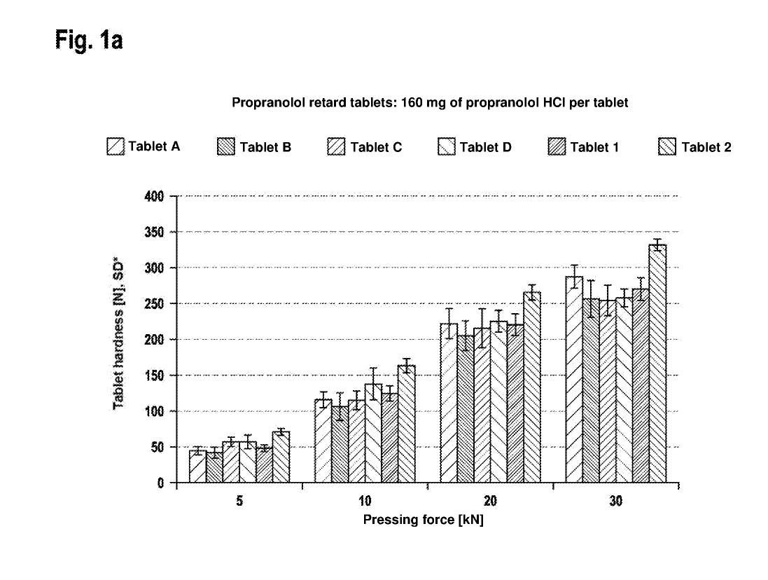

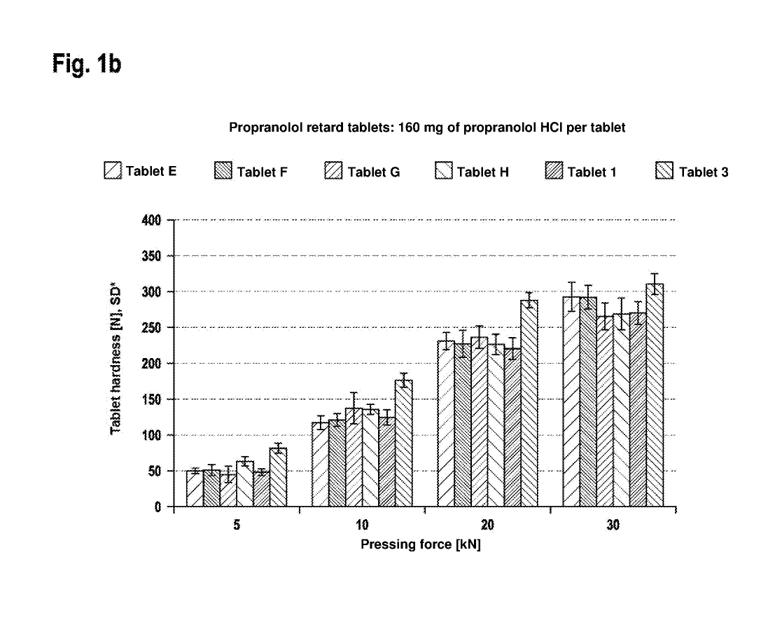

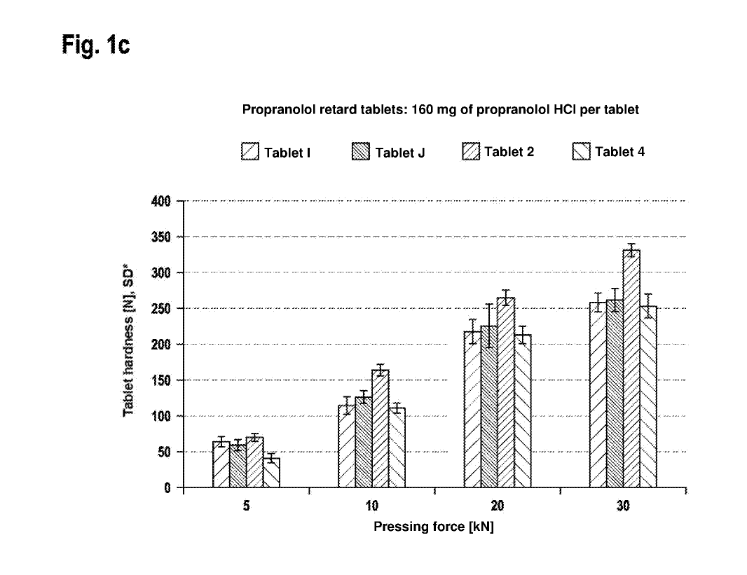

FIG. 1a shows a graph of the pressing force/tablet hardness profiles of the examples and Comparisons for better illustration.

### FIG. 1a: Pressing Force/Tablet Hardness Profiles of Propranolol HCl Retard Tablets A to D and 1 and 2

(*: SD: standard deviation)

TABLE 4b
Tableting data of propranolol HCl retard tablets using the
pre-mixtures of Examples E to H and Comparisons 1 and 3

| | A | | | | | | |
|---|---|---|---|---|---|---|---|
| Parameter | Nominal | Actual | B | C | D | E | F |
| Tablet E | 5 | 5.2 | 51 | 492.0 | 5.4 | 0.97 | 259 |
| | 10 | 9.9 | 118 | 496.7 | 4.9 | 0.12 | 395 |
| | 20 | 19.9 | 231 | 496.4 | 4.5 | 0.05 | 361 |
| | 30 | 31.2 | 293 | 494.1 | 4.4 | 0.06 | 349 |
| Tablet F | 5 | 4.9 | 55 | 502.2 | 5.5 | 0.92 | 176 |
| | 10 | 9.3 | 121 | 495.5 | 4.9 | 0.14 | 319 |
| | 20 | 19.6 | 228 | 492.8 | 4.5 | 0.06 | 354 |
| | 30 | 31.4 | 292 | 498.8 | 4.5 | 0.06 | 356 |
| Tablet G | 5 | 3.9 | 45 | 491.1 | 5.5 | 3.30 | 155 |
| | 10 | 11.1 | 138 | 493.6 | 4.8 | 0.06 | 290 |
| | 20 | 21.1 | 237 | 509.8 | 4.6 | 0.02 | 316 |
| | 30 | 30.1 | 266 | 502.0 | 4.5 | 0.00 | 311 |
| Tablet H | 5 | 5.3 | 63 | 502.0 | 5.4 | 0.70 | 194 |
| | 10 | 10.4 | 136 | 500.1 | 4.9 | 0.09 | 264 |
| | 20 | 18.9 | 227 | 498.7 | 4.6 | 0.06 | 275 |
| | 30 | 29.9 | 269 | 498.8 | 4.6 | 0.03 | 275 |
| Tablet 1 | 5 | 4.7 | 48 | 499.7 | 5.3 | 1.09 | 160 |
| | 10 | 10.1 | 125 | 504.3 | 4.8 | 0.01 | 267 |
| | 20 | 19.8 | 220 | 501.5 | 4.4 | 0.00 | 311 |
| | 30 | 28.8 | 270 | 506.2 | 4.4 | 0.01 | 322 |
| Tablet 3 | 5 | 5.4 | 82 | 448.2 | 4.7 | 0.15 | 507 |
| | 10 | 10.3 | 177 | 451.6 | 4.2 | 0.03 | 556 |
| | 20 | 22.2 | 288 | 455.8 | 4.0 | 0.03 | 443 |
| | 30 | 29.4 | 311 | 454.1 | 4.0 | 0.00 | 439 |

Parameters:
A: Pressing force [kN]
B: Tablet hardness after 1 day [N]
C: Tablet weight [mg]
D: Tablet height [mm]
E: Abrasion [%]
F: Ejection force (N)

FIG. 1b shows a graph of the pressing force/tablet hardness profiles of the examples and Comparisons for better illustration.

### FIG. 1b: Pressing Force/Tablet Hardness Profiles of Propranolol HCl Retard Tablets E to H and 1 and 3

TABLE 4c
Tableting data of propranolol HCl retard tablets using the
pre-mixtures of Examples I and J and Comparisons 2 and 4

| | A | | | | | | |
|---|---|---|---|---|---|---|---|
| Parameter | Nominal Actual | | B | C | D | E | F |
| Tablet I | 5 | 5.6 | 64 | 496.9 | 5.2 | 0.45 | 238 |
| | 10 | 9.8 | 115 | 500.2 | 4.9 | 0.09 | 334 |
| | 20 | 19.5 | 218 | 502.4 | 4.6 | 0.06 | 344 |
| | 30 | 28.9 | 258 | 500.7 | 4.5 | 0.05 | 340 |
| Tablet J | 5 | 5.4 | 59 | 506.9 | 5.4 | 0.94 | 209 |
| | 10 | 10.6 | 126 | 506.6 | 5.0 | 0.18 | 312 |
| | 20 | 20.7 | 226 | 505.9 | 4.7 | 0.08 | 307 |
| | 30 | 31.2 | 262 | 507.6 | 4.6 | 0.08 | 302 |
| Tablet 2 | 5 | 5.1 | 71 | 449.2 | 4.8 | 0.28 | 298 |
| | 10 | 9.9 | 164 | 451.1 | 4.3 | 0.03 | 404 |
| | 20 | 18.6 | 265 | 450.0 | 4.0 | 0.04 | 370 |
| | 30 | 32.2 | 332 | 453.3 | 4.0 | 0.04 | 376 |
| Tablet 4 | 5 | 4.5 | 42 | 493.1 | 5.4 | 1.57 | 187 |
| | 10 | 9.9 | 111 | 501.6 | 4.9 | 0.12 | 388 |
| | 20 | 19.3 | 213 | 500.7 | 4.5 | 0.05 | 390 |
| | 30 | 29.1 | 254 | 496.4 | 4.4 | 0.05 | 381 |

Parameters:
A: Pressing force [kN]
B: Tablet hardness after 1 day [N]
C: Tablet weight [mg]
D: Tablet height [mm]
E: Abrasion [%]
F: Ejection force (N)

FIG. c shows a graph of the pressing force/tablet hardness profiles of the examples and Comparisons for better illustration.

### FIG. 1c: Pressing Force/Tablet Hardness Profiles of Propranolol HCl Retard Tablets I and J and 2 and 4

All co-mixtures exhibit good compressibility, where the tablets obtained, pressed at 10 to 30 kN, have high tablet hardnesses together with very low abrasion after mechanical loading (low friability).

There are virtually no differences in the tableting data between the tablets based on the matrices PVA 26-88 or PVA 40-88 or in combinations thereof with HPMC K100M or K4M. In particular, the tablet hardnesses are virtually identical at the same pressing forces.

Owing to the low bulk and tapped densities of Comparisons 2 and 3 (without PVA), the tablets of Comparisons 2 and 3 can only be pressed to a final weight of 450 mg.

### For Step 3: In Vitro Release of Propranolol HCl from the Retard Tablets Pressed at a Pressing Force of 20 kN in Phosphate Buffer pH 6.8 Over 12 or 42 Hours

(*: SD: standard deviation; **: Av: average)

TABLE 5a
In vitro release data of Examples A to D and Comparisons 1 and 2 at pH 6.8

| Time | Tablet A | | Tablet B | | Tablet C | | Tablet D | | Tablet 1 | | Tablet 2 | |
|---|---|---|---|---|---|---|---|---|---|---|---|---|
| (hours) | SD* | Av** | SD* | Av** | SD* | Av** | SD* | Av** | SD* | Av** | SD* | Av** |
| 1 | 0.5 | 14 | 0.3 | 13 | 0.2 | 12 | 0.1 | 10 | 0.4 | 15 | 0.3 | 11 |
| 2 | 1.1 | 21 | 0.6 | 20 | 0.3 | 18 | 0.2 | 15 | 0.7 | 26 | 0.4 | 16 |
| 3 | 1.6 | 28 | 1.0 | 26 | 0.4 | 23 | 0.2 | 20 | 1.1 | 35 | 0.5 | 20 |
| 4 | 2.1 | 34 | 1.4 | 32 | 0.5 | 27 | 0.2 | 23 | 1.3 | 43 | 0.6 | 24 |
| 5 | 2.6 | 40 | 1.7 | 37 | 0.6 | 31 | 0.2 | 27 | 1.6 | 50 | 0.7 | 27 |
| 6 | 3.1 | 45 | 2.0 | 42 | 0.7 | 35 | 0.2 | 30 | 1.8 | 57 | 0.8 | 30 |
| 8 | 4.0 | 54 | 2.7 | 51 | 0.8 | 42 | 0.3 | 37 | 2.3 | 70 | 1.0 | 35 |
| 10 | 4.8 | 63 | 3.3 | 59 | 0.9 | 49 | 0.3 | 42 | 2.3 | 81 | 1.1 | 41 |
| 12 | 5.4 | 70 | 3.9 | 66 | 0.9 | 55 | 0.4 | 48 | 1.8 | 89 | 1.2 | 46 |
| 17 | 5.3 | 85 | 5.7 | 82 | 0.9 | 67 | 0.6 | 60 | 1.1 | 96 | 1.3 | 59 |
| 22 | 2.7 | 93 | 6.3 | 93 | 0.8 | 78 | 1.0 | 70 | 0.8 | 99 | 1.2 | 70 |
| 27 | 1.3 | 98 | 4.2 | 98 | 0.8 | 86 | 1.4 | 79 | 0.5 | 101 | 1.1 | 79 |
| 32 | 0.8 | 100 | 2.1 | 101 | 0.9 | 92 | 1.7 | 86 | 0.2 | 102 | 1.0 | 86 |
| 37 | 0.9 | 102 | 0.8 | 102 | 1.1 | 97 | 1.9 | 93 | 0.0 | 102 | 0.9 | 93 |
| 42 | 1.1 | 103 | 0.4 | 103 | 0.6 | 101 | 2.0 | 98 | 0.0 | 103 | 0.8 | 98 |

The table shows the cumulative amounts of propranolol HCl (in %) released from the tablets obtained at a pressing force of 20 kN over 42 hours.

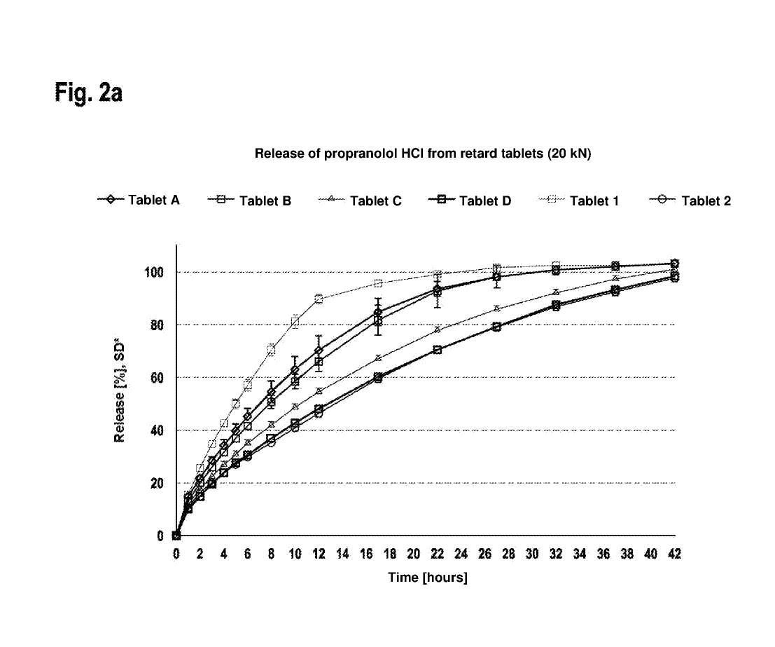

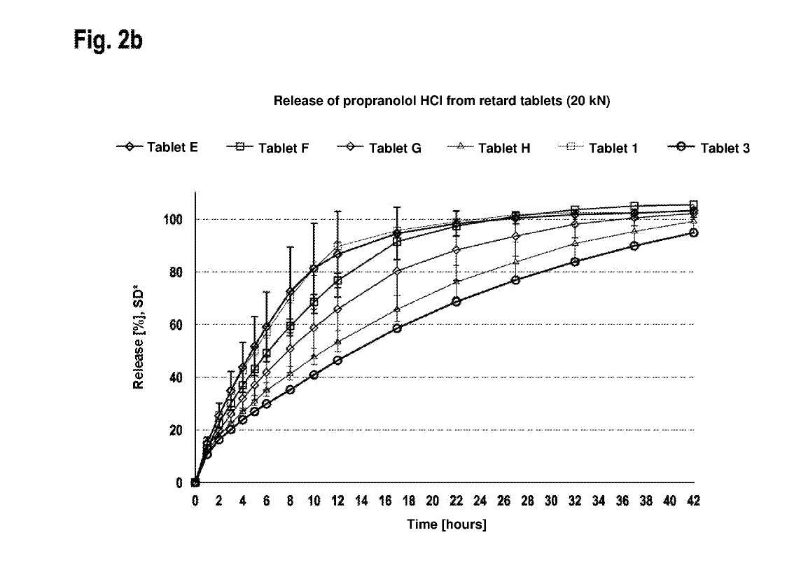

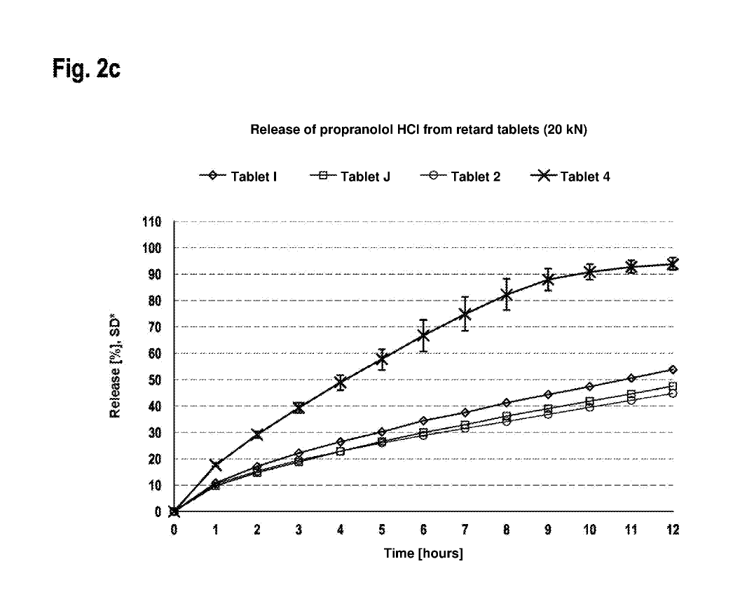

FIG. 2a shows a graph of the releases at pH 6.8 from Table 5a for better illustration.

### FIG. 2a: In-Vitro Release Data of the Tablets from Experiments A to D and 1 and 2 at pH 6.8 Over 42 Hours

TABLE 5b
In-vitro release data of Examples E to H and Comparisons 1 and 3 at pH 6.8

| Time | Tablet E | | Tablet F | | Tablet G | | Tablet H | | Tablet 1 | | Tablet 3 | |
|---|---|---|---|---|---|---|---|---|---|---|---|---|
| (hours) | SD* | Av** | SD* | Av** | SD* | Av** | SD* | Av** | SD* | Av** | SD* | Av** |
| 1 | 1.9 | 15 | 0.1 | 14 | 0.9 | 12 | 0.4 | 11 | 0.4 | 15 | 0.2 | 11 |
| 2 | 4.3 | 26 | 0.2 | 23 | 2.1 | 20 | 0.7 | 17 | 0.7 | 26 | 0.3 | 16 |
| 3 | 6.7 | 35 | 0.4 | 30 | 3.3 | 26 | 1.0 | 22 | 1.1 | 35 | 0.3 | 20 |
| 4 | 9.1 | 44 | 0.6 | 37 | 4.5 | 32 | 1.4 | 27 | 1.3 | 43 | 0.3 | 23 |
| 5 | 11.1 | 52 | 0.9 | 43 | 5.6 | 37 | 1.8 | 31 | 1.6 | 50 | 0.4 | 27 |
| 6 | 13.1 | 59 | 1.2 | 49 | 6.8 | 42 | 2.1 | 35 | 1.8 | 57 | 0.4 | 29 |
| 8 | 16.7 | 72 | 2.0 | 59 | 8.8 | 51 | 2.7 | 42 | 2.3 | 70 | 0.6 | 35 |
| 10 | 17.4 | 81 | 2.5 | 69 | 10.7 | 59 | 3.3 | 48 | 2.3 | 81 | 0.7 | 41 |
| 12 | 16.2 | 87 | 2.8 | 76 | 12.7 | 66 | 3.8 | 54 | 1.8 | 89 | 0.8 | 46 |
| 17 | 10.4 | 94 | 1.6 | 91 | 14.7 | 80 | 4.9 | 66 | 1.1 | 96 | 0.9 | 58 |
| 22 | 4.6 | 98 | 0.5 | 98 | 11.6 | 88 | 5.9 | 76 | 0.8 | 99 | 0.8 | 68 |
| 27 | 2.1 | 100 | 0.3 | 101 | 7.6 | 93 | 6.7 | 84 | 0.5 | 101 | 0.8 | 77 |
| 32 | 1.0 | 101 | 0.7 | 103 | 5.1 | 98 | 7.4 | 91 | 0.2 | 102 | 0.8 | 84 |
| 37 | 0.5 | 102 | 1.0 | 105 | 2.8 | 100 | 6.3 | 95 | 0.0 | 102 | 0.5 | 90 |
| 42 | 0.2 | 103 | 1.2 | 106 | 1.2 | 102 | 4.4 | 99 | 0.0 | 103 | 0.4 | 95 |

The table shows the cumulative amounts of propranolol HCl (in %) released from the tablets obtained at a pressing force of 20 kN over 42 hours.

FIG. 2b shows a graph of the release data at pH 6.8 from Table 5b for better illustration.

### FIG. 2b: In-Vitro Release Data of the Tablets of Examples E to H and Comparisons 1 and 3 at pH 6.8 Over 42 Hours

TABLE 5c
In-vitro release data of Examples RA and
J and Comparisons 2 and 4 at pH 6.8

| Time | Tablet I | | Tablet J | | Tablet 4 | | Tablet 2 | |
|---|---|---|---|---|---|---|---|---|
| (hours) | SD* | Av** | SD* | Av** | SD* | Av** | SD* | Av** |
| 1 | 0.0 | 11 | 0.1 | 10 | 0.6 | 18 | 0.1 | 11 |
| 2 | 0.1 | 17 | 0.3 | 15 | 1.2 | 29 | 0.2 | 16 |
| 3 | 0.2 | 22 | 0.4 | 19 | 2.0 | 39 | 0.2 | 19 |
| 4 | 0.2 | 26 | 0.6 | 23 | 2.9 | 49 | 0.3 | 23 |
| 5 | 0.3 | 31 | 0.7 | 27 | 3.9 | 58 | 0.3 | 26 |
| 6 | 0.4 | 34 | 0.9 | 30 | 5.9 | 67 | 0.3 | 29 |
| 7 | 0.4 | 38 | 1.1 | 33 | 6.3 | 75 | 0.3 | 31 |
| 8 | 0.5 | 41 | 1.2 | 36 | 5.9 | 82 | 0.3 | 34 |
| 9 | 0.6 | 45 | 1.3 | 39 | 4.2 | 88 | 0.3 | 37 |
| 10 | 0.7 | 48 | 1.4 | 42 | 2.9 | 91 | 0.2 | 40 |
| 11 | 0.7 | 51 | 1.5 | 45 | 2.5 | 93 | 0.1 | 42 |
| 12 | 0.8 | 54 | 1.6 | 47 | 2.2 | 94 | 0.0 | 45 |

The table shows the cumulative amounts of propranolol HCl (in %) released from the tablets obtained at a pressing force of 20 kN over 12 hours.

FIG. 2c shows a graph of the release data at pH 6.8 FROM Table 5c for better illustration.

### FIG. 2c: In-Vitro Release Data of Tablets I to J and 2 and 4 at pH 6.8 Over 12 Hours

### Conclusion

1. All Examples A to J of the retardation matrices based on PVA (irrespective of whether PVA 26-88 or PVA 40-88 with HPMC K100M or HPMC K4M) clearly show higher bulk and tapped densities than the matrices comprising HPMC K100M or HPMC K4M without PVA. This property allows the formulation of retard tablets having smaller dimensions for the same tablet weight.

2. The added amounts of HPMC do not result in impairment of the compressibility—all mixtures are suitable for use in direct compression processes.

3. With small amounts of HPMC added to the PVA-containing mixtures, the in vitro release behaviour of propranolol can be significantly slowed or extended. Even with only 4.5 to 15% by weight of the HPMC grades of different viscosity employed in the co-mixtures, widely differing in vitro release profiles can, depending on the present need of the developer, be modulated and also extended significantly beyond 12 hours.

1. Directly compressible co-mixtures for the preparation of pharmaceutical formulations, comprising finely divided polyvinyl alcohols (PVAs) and finally divided, microcrystalline celluloses (MCCs) in combination with finely divided hydroxypropylmethylcelluloses (HPMCs).

2. Directly compressible co-mixtures according to claim 1 which have bulk densities in the range from 0.35 to 0.45 g/ml.

3. Directly compressible co-mixtures according to claim 1, having a tapped density in the range from 0.53 to 0.63 g/ml.

4. Directly compressible co-mixtures according to claim 1, comprising finely divided polyvinyl alcohols (PVAs), finally divided microcrystalline celluloses (MCCs) and finely divided hydroxypropylmethylcelluloses (HPMCs), which have a weight ratio to one another in the mixture in the range from 50:45.5:4.5 to 50:15:35.

5. Directly compressible co-mixtures according to claim 1, for the preparation of formulations having particularly extended release of an active pharmaceutical ingredient, in which the release duration of the active pharmaceutical ingredient is controlled by the ratio of the components to one another in the co-mixture.

6. Directly compressible co-mixtures according to claim 1, in which the release duration of the active pharmaceutical ingredient is controlled by the amount of HPMC present in the co-mixture.

7. Preparation of co-mixtures according to claim 1, characterised in that ground PVAs in pharmaceutical grade, in particular in pharmacopoeia grade, are used.

8. Preparation of co-mixtures according to claim 7, characterised in that ground, finely divided PVAs having average particle sizes in the range from 40 to 120 μm, in particular in the range from 70 to 90 μm, are used.

9. Preparation of co-mixtures according to claim 7, characterised in that use is made of ground, finely divided PVAs selected from the group of grades 18-88, 26-88, 40-88 and 28-99, preferably from the group 26-88 and 40-88.

10. Preparation of co-mixtures according to claim 7, characterised in that an HPMC in pharmaceutical grade, in particular in a pharmacopoeia grade, is used.

11. Preparation of co-mixtures according to claim 7, characterised in that an HPMC selected from the group of grades K100M and K4M, or an HPMC grade which is between these two grades with respect to its viscosity, is used.

12. (canceled)

13. Tablets containing active pharmaceutical ingredient having extended release of active pharmaceutical ingredient of more than 12 hours, comprising a co-mixture of finely divided PVA, finely divided MCC and finely divided HPMC according to claim 1.

14. Tablets containing active pharmaceutical ingredient according to claim 13, comprising the directly compressible co-mixture in an amount in the range from 1-99% by weight, based on the total weight of the tablet.

15. Tablets containing active pharmaceutical ingredient according to claim 13 which have been produced using low pressing forces and have particularly high tablet hardnesses at the same time as low friabilities of =/<0.2% by weight.

16. Tablets containing active pharmaceutical ingredient according to claim 13 having extended release of active pharmaceutical ingredient, comprising active pharmaceutical ingredients from BCS class I, either alone or in combination with other active pharmaceutical ingredients.
